(19) United States
(12) Patent Application Publication
(10) Pub. No.: US 2024/0374436 A1
(43) Pub. Date: Nov. 14, 2024

(54) Infant Hernia Bandage Device

(71) Applicant: David Phillips, JR., Amite, LA (US)

(72) Inventor: David Phillips, JR., Amite, LA (US)

(21) Appl. No.: 18/635,170

(22) Filed: Apr. 15, 2024

Related U.S. Application Data
(60) Provisional application No. 63/464,956, filed on May 9, 2023.

(51) Int. Cl. A61F 13/0246

(52) U.S. Cl. CPC A61F 13/0253

(57) ABSTRACT

An infant hernia bandage device is provided. The device is comprised of an infant hernia bandage device that can be placed over a hernia area on the body of an infant via an adhesive on a bottom surface of the device to compress the hernia area. The top surface of the device is comprised of a padding that protects the hernia area from impacts. During use, the device can be used in conjunction with a diaper to further compress the hernia area by placing a strap of the diaper over the position of the bandage on an infant.

### CROSS-REFERENCE TO RELATED APPLICATION

The present application claims priority to, and the benefit of, U.S. Provisional Application No. 63/464,956, which was filed on May 9, 2023, and is incorporated herein by reference in its entirety.

### FIELD OF THE INVENTION

The present invention relates generally to the field of hernia treatment devices. More specifically, the present invention relates to an infant hernia bandage device that can be applied to an infant's body to compress a hernia area, and wherein a padding of the device protects the hernia area from impacts. Accordingly, the present disclosure makes specific reference thereto. Nonetheless, it is to be appreciated that aspects of the present invention are also equally applicable to other like applications, devices, and methods of manufacture.

### BACKGROUND

An inguinal hernia occurs when tissue, such as part of the intestine, protrudes through a weak spot in the abdominal muscles. Infants that suffer from inguinal hernias can be incredibly tender and uncomfortable. This may result in excessive crying, vomiting, bowel movement discomfort, and irregular eating habits due to the discomfort. Hernia bulge can hinder children and prevent them from laying on their stomachs or crawling. However, existing devices do not allow for a hernia bulge to be sufficiently protected, especially in infants.

Therefore, there exists a long-felt need in the art for a hernia device. More specifically, there exists a long-felt need in the art for a hernia bandage device that can be used to protect a hernia bulge. In addition, there exists a long-felt need in the art for an infant hernia bandage device that protects a hernia bulge in an infant. Further, there exists a long-felt need in the art for an infant hernia bandage device that protects a hernia bulge on an infant and prevents the hernia from protruding.

The subject matter disclosed and claimed herein, in one embodiment thereof, comprises an infant hernia bandage device. The device is comprised of an infant hernia bandage device. The device is designed to be placed over a hernia area on the body of an infant via an adhesive on a bottom surface of the device to compress the hernia area. The top surface of the device is comprised of a padding that protects the hernia area from impacts. The present invention may also include a method of using the device in conjunction with a diaper to further compress the hernia area by placing a strap of the diaper over the position of the bandage on an infant.

In this manner, the infant hernia bandage device of the present invention accomplishes all the foregoing objectives and provides a device that can be used to protect a hernia bulge. In addition, the device protects a hernia bulge in an infant. Furthermore, the device prevents the hernia from protruding during use.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises an infant hernia bandage device. The device is primarily comprised of a body with an adhesive bottom surface and a foam padding. During use, the device is designed to be applied to the skin of an infant, over the area of an inguinal hernia, such that the device compresses said area and the diaper strap of the infant can be positioned over the foam padding to provide further compression.

The body of the device may be any size and shape such that it can cover a full hernia area of an infant. The bottom surface of the body is comprised of a skin-safe adhesive. The adhesive secures the body on the infant's skin over the hernia area.

The top surface of the body is comprised of at least one foam padding that protects the hernia area from impacts. The padding may be made from any foam or other padding known in the art. The padding may cover the entire top surface or only a portion of the top surface.

In one embodiment, the top surface is also comprised of at least one fastener. The fastener may be found on the top surface (in an embodiment wherein the padding does not cover the entire top surface) or on the top surface of the padding. The fastener is preferably an adhesive fastener (or any fastener of the like) that allows the device to also attach to the interior of the diaper of the infant. In this manner, the device is further secured in position.

The present invention is also comprised of a method of using the device. First, a device is provided comprised of a body with an adhesive bottom surface and a foam padding on the top surface. Then, the bottom surface is adhered to the skin of an infant, specifically over the hernia area of the infant's body. Next, the diaper is placed over the infant's lower body while covering the device. Finally, the diaper strap is used to secure the diaper to the infant, wherein the strap is specifically positioned tightly over the device to further compress the hernia area to compress the hernia.

The present invention is also comprised of a second method of using the device. First, a device is provided comprised of a body with an adhesive bottom surface and a foam padding on the top surface, wherein the padding and/or top surface is comprised of at least one fastener. Then, the bottom surface is adhered to the skin of an infant, specifically over the hernia area of the infant's body. Next, the diaper is placed over the infant's lower body, wherein the fastener is secured to the diaper to prevent movement of the diaper and device. Finally, the diaper strap is used to secure the diaper to the infant, wherein the strap is specifically positioned tightly over the device to further compress the hernia area to compress the hernia.

Accordingly, the infant hernia bandage device of the present invention is particularly advantageous as it provides a device that can be used to protect a hernia bulge. More specifically, the device protects a hernia bulge in an infant. Furthermore, the device prevents the hernia from protruding during use. In this manner, the infant hernia bandage device provides a solution to protecting infant hernias.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a top view of one potential embodiment of an infant hernia bandage device of the present invention while attached over a hernia area of an infant in accordance with the disclosed architecture;

FIG. 2 illustrates a side view of one potential embodiment of an infant hernia bandage device of the present invention in accordance with the disclosed architecture;

FIG. 3 illustrates a top view of one potential embodiment of an infant hernia bandage device of the present invention while attached over a hernia area of an infant wearing a diaper in accordance with the disclosed architecture;

FIG. 4 illustrates a flowchart of a method of using one potential embodiment of an infant hernia bandage device of the present invention in accordance with the disclosed architecture; and

FIG. 5 illustrates a flowchart of a method of using one potential embodiment of an infant hernia bandage device of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for a hernia bandage device that can be used to protect a hernia bulge. In addition, there exists a long-felt need in the art for an infant hernia bandage device that protects a hernia bulge in an infant. Further, there exists a long-felt need in the art for an infant hernia bandage device that protects a hernia bulge on an infant and prevents the hernia from protruding.

The present invention, in one exemplary embodiment, is comprised of an infant hernia bandage device primarily comprised of a body with an adhesive bottom surface and a foam padding. During use, the device can be applied to the skin of an infant, specifically over the area of an inguinal hernia, such that the device compresses said area, and the diaper strap of the infant can be positioned over the foam padding to provide further compression.

The body of the device may be any size and shape such that it can cover a full hernia area of an infant. The bottom surface of the body is comprised of a skin-safe adhesive that secures the body on the infant's skin over the hernia area. The top surface of the body is comprised of at least one foam padding that protects the hernia area from impacts. The padding may be made from any foam or other padding known in the art. The padding may cover the entire top surface or only a portion of the top surface.

The top surface may also be comprised of at least one fastener. The fastener may be found on the top surface (in an embodiment wherein the padding does not cover the entire top surface) or on the top surface of the padding. The fastener is preferably an adhesive fastener (or any fastener of the like) that allows the device to also attach to the interior of the diaper of the infant. In this manner, the device is further secured in position.

The present invention is also comprised of a method of using the device. First, a device is provided comprised of a body with an adhesive bottom surface and a foam padding on the top surface. Then, the bottom surface is adhered to the skin of an infant, specifically over the hernia area of the infant's body. Next, the diaper is placed over the infant's lower body while covering the device. Finally, the diaper strap is used to secure the diaper to the infant, wherein the strap is specifically positioned tightly over the device to further compress the hernia area to compress the hernia.

The present invention is also comprised of a second method of using the device. First, a device is provided comprised of a body with an adhesive bottom surface and a foam padding on the top surface, wherein the padding and/or top surface is comprised of at least one fastener. Then, the bottom surface is adhered to the skin of an infant, specifically over the hernia area of the infant's body. Next, the diaper is placed over the infant's lower body, wherein the fastener is secured to the diaper to prevent movement of the diaper and device. Finally, the diaper strap is used to secure the diaper to the infant, wherein the strap is specifically positioned tightly over the device to further compress the hernia area to compress the hernia.

Accordingly, the infant hernia bandage device of the present invention is particularly advantageous as it provides a device that can be used to protect a hernia bulge. More specifically, the device protects a hernia bulge in an infant. Furthermore, the device prevents the hernia from protruding during use. In this manner, the infant hernia bandage device provides a solution to protecting infant hernias.

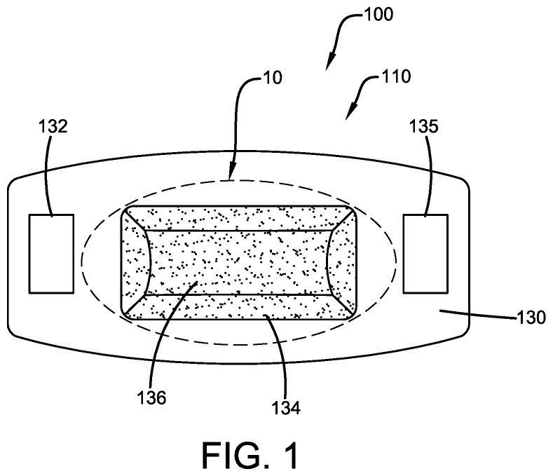

Referring initially to the drawings, FIG. 1 illustrates a top view of one potential embodiment of an infant hernia bandage device 100 of the present invention while attached over a hernia area 10 of an infant in accordance with the disclosed architecture. The device 100 is primarily comprised of a body 110 with an adhesive 122 bottom surface 120 and a foam padding 134. During use, the device 100 is designed to be applied to the skin of an infant to compress the area of an inguinal hernia 10, and wherein a diaper strap 14 of the infant's diaper can be positioned over the foam padding 134 such that the hernia area 10 is further compressed.

The body 110 of the device 100 may be any size and shape such that it can cover a full hernia area 10 of an infant. The body 110 may be any color and have any pattern/design. In one embodiment, the body 110 is transparent to aid in the placement of the device 100 over the hernia area 10. The body 110 is preferably made from a woven fabric, a plastic, or a latex material. The body 110 may or may not be waterproof (for bodily fluid protection).

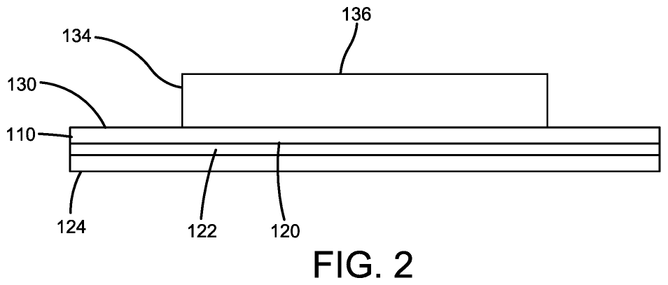

The bottom surface 120 of the body 110 is comprised of a skin-safe adhesive 122, as seen in FIG. 2. The adhesive is preferably comprised of an acrylate adhesive, but may be any skin-safe bandage adhesive known in the art. The adhesive 122 secures the body 110 on the infant's skin over the hernia area 10. The adhesive 122 is covered by a peelable cover/film 124 when not in use.

The top surface 130 of the body 110 is comprised of at least one foam padding 134. The padding 134 protects the hernia area 10 from impacts. The padding 134 may be made from any foam or other padding known in the art. The padding 134 may cover the entire top surface 130 or only a portion of the top surface 130. The padding 134 may have a flat top edge 136 or a convex top edge 136 for further protection. The padding 134 may also be comprised of at least one weighted element 135 such as but not limited to a copper weighted element weighting approximately (but not limited to 2.50 grams).

In one embodiment, the top surface 130 is also comprised of at least one fastener 132. The fastener 132 may be found on the top surface 130 (in an embodiment wherein the padding 134 does not cover the entire top surface 130) or on the top surface 133 of the padding 134. The fastener 132 is preferably an adhesive fastener (or any fastener of the like) that allows the device 100 to also attach to the interior of the diaper 12 of the infant. In this manner, the device 100 is further secured in position.

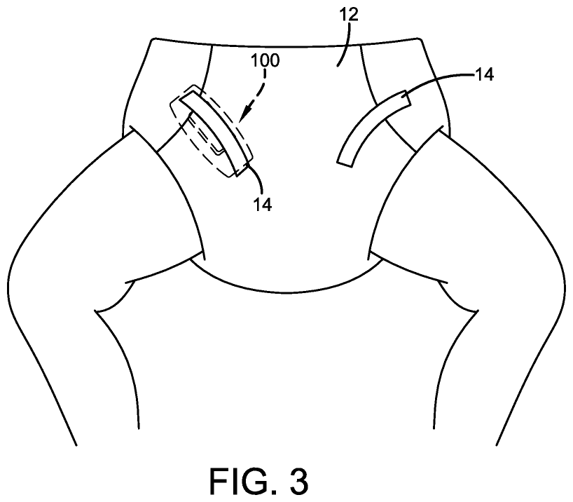

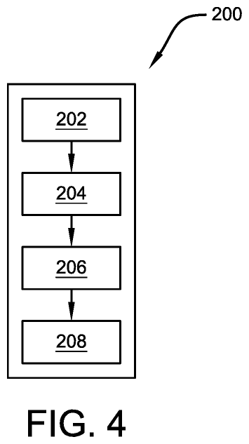

The present invention is also comprised of a method of using 200 the device 100, as seen in FIG. 4. First, a device 100 is provided comprised of a body with an adhesive 122 bottom surface 120 and a foam padding 134 on the top surface 130 [Step 202]. Then, the bottom surface 120 is adhered to the skin of an infant, specifically over the hernia area 10 of the infant's body [Step 204]. Next, the diaper 12 is placed over the infant's lower body while covering the device 100 [Step 206]. Finally, the diaper strap 14 is used to secure the diaper 12 to the infant, wherein the strap 14 is specifically positioned tightly over the device 100 to further compress the hernia area 10 to compress the hernia, as seen in FIG. 3 [Step 208].

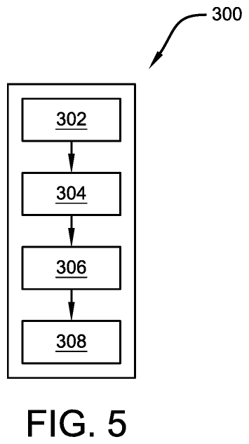

The present invention is also comprised of a second method of using 300 the device 100, as seen in FIG. 5. First, a device 100 is provided comprised of a body with an adhesive 122 bottom surface 120 and a foam padding 134 on the top surface 130, wherein the padding 134 and/or top surface 130 is comprised of at least one fastener 132 [Step 302]. Then, the bottom surface 120 is adhered to the skin of an infant, specifically over the hernia area 10 of the infant's body [Step 304]. Next, the diaper 12 is placed over the infant's lower body, wherein the fastener 132 is secured to the diaper 12 to prevent movement of the diaper 12 and device 100 [Step 206]. Finally, the diaper strap 14 is used to secure the diaper 12 to the infant, wherein the strap 14 is specifically positioned tightly over the device 100 to further compress the hernia area 10 to compress the hernia [Step 308].

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “infant hernia bandage device” and “device” are interchangeable and refer to the infant hernia bandage device 100 of the present invention.

Notwithstanding the foregoing, the infant hernia bandage device 100 of the present invention and its various components can be of any suitable size and configuration as is known in the art without affecting the overall concept of the invention, provided that they accomplish the above-stated objectives. One of ordinary skill in the art will appreciate that the size, configuration, and material of the infant hernia bandage device 100 as shown in the FIGS. are for illustrative purposes only, and that many other sizes and shapes of the infant hernia bandage device 100 are well within the scope of the present disclosure. Although the dimensions of the infant hernia bandage device 100 are important design parameters for user convenience, the infant hernia bandage device 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An infant hernia bandage device comprising:
a body comprised of a top surface and a bottom surface;
an adhesive positioned on the bottom surface; and
a padding positioned on the top surface.

2. The infant hernia bandage device of claim 1, wherein the body is comprised of a woven fabric.

3. The infant hernia bandage device of claim 1, wherein the body is comprised of a plastic.

4. The infant hernia bandage device of claim 1, wherein the body is comprised of a latex.

5. The infant hernia bandage device of claim 1, wherein the body is comprised of a waterproof material.

6. The infant hernia bandage device of claim 1, wherein the body is transparent.

7. The infant hernia bandage device of claim 1, wherein the adhesive is comprised of an acrylate adhesive.

8. The infant hernia bandage device of claim 1, wherein the padding is comprised of a foam padding.

9. The infant hernia bandage device of claim 1, wherein the foam padding covers the entirety of the top surface.

10. An infant hernia bandage device comprising:
a body comprised of a top surface and a bottom surface;
an adhesive positioned on the bottom surface;
a padding positioned on the top surface; and
a fastener.

11. The infant hernia bandage device of claim 10, wherein the padding is comprised of a foam padding.

12. The infant hernia bandage device of claim 10, wherein the fastener is positioned on the top surface.

13. The infant hernia bandage device of claim 12, wherein the fastener is comprised of an adhesive.

14. The infant hernia bandage device of claim 10, wherein the fastener is positioned on a top surface of the padding.

15. The infant hernia bandage device of claim 14, wherein the fastener is comprised of an adhesive.

16. The infant hernia bandage device of claim 10, wherein the padding is comprised of a convex top surface.

17. The infant hernia bandage device of claim 13, wherein the adhesive is comprised of a skin-safe adhesive.

18. A method of using an infant hernia bandage device, the method comprising the following steps:
providing an infant hernia bandage device comprised of a body with a bottom surface comprised of an adhesive and a top surface comprised of a foam padding;
adhering the bottom surface to a hernia area on an infant's body;
placing a diaper over the infant's lower body such that the diaper covers the infant hernia bandage device; and
positioning a strap of the diaper over an area of the diaper the device is positioned under to further compress the hernia area.

19. The method of claim 18, wherein the top surfaced is comprised of a fastener.

20. The method of claim 19 further comprised of a step of securing the fastener to the diaper.
